(19) United States
(12) Patent Application Publication
(10) Pub. No.: US 2008/0146923 A1
(43) Pub. Date: Jun. 19, 2008

(54) Composite ultrasound 3D intracardiac volume by aggregation of individual ultrasound 3D intracardiac segments

(75) Inventors: Claudio Patricio Mejia, Wauwatosa, WI (US); Sachin Vadodaria, Fox Point, WI (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/607,744

(22) Filed: Dec. 1, 2006

Related U.S. Application Data
(60) Provisional application No. 60/853,108, filed on Oct. 20, 2006.

(51) Int. Cl. A61B 8/00

(52) U.S. Cl. 600/437

(57) ABSTRACT

Embodiments of the presently described technology provide a method for ultrasound imaging. The method includes obtaining a plurality of ultrasound 3D image segments of an anatomy and combining the plurality of 3D image segments into a composite 3D image of the anatomy. Embodiments of the presently described technology also provide a system for ultrasound imaging. The system includes a computing device combining a plurality of ultrasound 2D images of an anatomy obtained by a transducer array into one or more 3D image segments and aggregating the 3D image segments into a composite 3D image of the anatomy.

### RELATED APPLICATIONS

This application claims the benefit of U.S. Provisional Application No. 60/853,108 (the “'108 application”), filed Oct. 20, 2006, entitled “Composite Ultrasound 3D Intracardiac Volume by Aggregation of Individual Ultrasound 3D Intracardiac Segments.” The '108 application is incorporated by reference herein in its entirety.

### BACKGROUND OF THE INVENTION

The presently described technology relates to ultrasound imaging. Specifically, embodiments of the presently described invention relate to improved systems and methods for three-dimensional (“3D”) ultrasound imaging.

Existing intracardiac echocardiography (“ICE”) ultrasound imaging technology provides a two-dimensional (“2D”) view of a patient anatomy, such as a patient's heart. This technology includes mounting a transducer array on the exterior of a catheter, inserting the transducer array into a patient's heart, activating the elements of the array to transmit and receive ultrasound echoes and translating or converting the received ultrasound echoes into a 2D image.

Some ICE ultrasound imaging technology also provides a 3D volume image of a patient anatomy, such as a patient's heart. However, the current systems and methods for providing such 3D volumes provide a very limited imaged volume.

Although these technologies allow clinicians to get an internal view of the cardiac anatomy and provide a means to deliver image guided therapy, it makes it difficult for the clinician to get an exact indication of where the ICE catheter is located. For example, 2D images typically do not provide sufficient information to determine the location of the catheter with respect to structures in the cardiac anatomy. In another example, 3D images obtained by existing ultrasound technologies provide a very limited volumetric image. In a sense, the existing 3D images are akin to shining a spotlight to view a large area. While the area illuminated by the spotlight can be viewed, other areas that are not illuminated cannot be viewed.

Thus, existing technologies do not provide sufficient imaging information about the volume surrounding the ICE catheter. One way to solve this problem could be to provide more 3D imaging information. That is, by providing a wider ultrasound-rendered volume of the cardiac anatomy and/or anatomical structure than currently available from existing technologies, clinicians can be able to better identify the catheter location with respect to the patient's anatomy. This volume could be rendered in real-time (or, created as additional imaging information/data is obtained by a transducer array) and provide immediate feedback to clinicians and accordingly assist in the determination of the ICE catheter location.

Therefore, a need exists for an improved system and method for providing an increased imaged volume using catheter-based ultrasound transducer arrays. Meeting such a need can provide clinicians with additional imaging information in patients' cardiac anatomies.

### BRIEF DESCRIPTION OF THE INVENTION

Embodiments of the presently described technology provide a method for ultrasound imaging. The method includes obtaining a plurality of ultrasound 3D image segments of an anatomy and combining the plurality of 3D image segments into a composite 3D image of the anatomy.

Embodiments of the presently described technology provide a system for ultrasound imaging. The system includes a computing device combining a plurality of ultrasound 2D images of an anatomy obtained by a transducer array into one or more 3D image segments and aggregating the 3D image segments into a composite 3D image of the anatomy.

Embodiments of the presently described technology provide a computer-readable storage medium comprising a set of instructions for a computing device. The set of instructions include an aggregation routine configured to aggregate a plurality of 3D ultrasound image segments into a composite 3D image of an anatomy, where the 3D image segments are formed by combining a plurality of 2D ultrasound images of an anatomy.

### BRIEF DESCRIPTION OF SEVERAL VIEWS OF THE DRAWINGS

FIG. 1 illustrates a catheter-based ultrasound imaging system according to an embodiment of the presently described technology.

FIG. 2 illustrates distal end of elongated body with a transducer tip in accordance with an embodiment of the presently described technology.

FIG. 3 illustrates a group of 3D image segments obtained in accordance with an embodiment of the presently described technology.

FIG. 4 illustrates a composite 3D image formed or created from a plurality of 3D image segments by computing device in accordance with an embodiment of the presently described technology.

FIG. 5 illustrates a flowchart of a method for aggregating a plurality of 3D image segments into a composite 3D image in accordance with an embodiment of the presently described technology.

The foregoing summary, as well as the following detailed description of certain embodiments of the presently described technology, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Embodiments of the presently described technology provide a mechanism visualize in 3D an intracardiac volume/anatomical structure. In addition, embodiments of the presently described technology provide a mechanism to a clinician to identify the location of an ultrasound imaging intracardiac echocardiographic catheter (that is, a point of view). Embodiments of the presently described technology also allow 3D volume to be rendered in real-time as 3D images are acquired or via post processing activity by aggregating previously acquired 3D ultrasound intracardiac segments.

Current ICE catheters can only provide 2D images. There is no 3D ICE catheter available in the marketplace that is being actively used by clinicians to diagnose and treat patients. Furthermore, there are no composite 3D intracardiac volumes that are being created using currently available catheter probes. Embodiments of the presently described technology extend the use of 3D ICE catheters by introducing the concept of aggregation of the acquired 3D ultrasound images to create a composite volume.

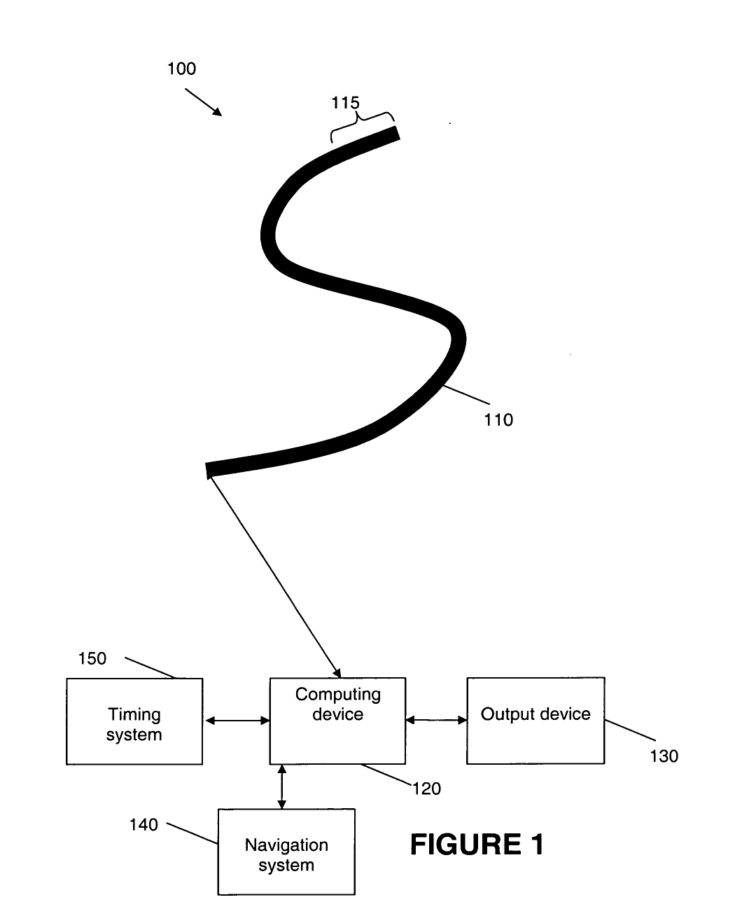

FIG. 1 illustrates a catheter-based ultrasound imaging system 100 according to an embodiment of the presently described technology. System 100 includes an elongated body 110, a computing device 120, an output device 130, a navigation system 140 and a timing system 150. Elongated body 110 is in communication with computing device 120. Computing device 120 is in communication with output device 130, navigation system 140 and timing system 150. Any one or more of elongated body 110, computing device 120 and output device 130 can communicate data via one or more digital connections. The digital connections can be a wired or wireless connection, for example.

Elongated body 110 includes any elongated device or apparatus capable of having a transducer array mounted proximate a distal end 115 of the body 110. For example, in a preferred embodiment, elongated body 110 includes a catheter. In other embodiments, elongated body 110 can include an endoscope, for example.

Computing device 120 includes any device capable of carrying out a set of instructions for a computer. For example, computing device 120 can include a CPU. In another example, computing device 120 can include an ultrasound imaging CPU. As described in more detail below, computing device 120 is capable of directing a transducer array to obtain 2D ultrasound images in a plurality of imaging planes in a patient anatomy. For example, computing device 120 can direct a linear phased transducer array to obtain one or more 2D images. Computing device 120 can direct which plane should be imaged and when an ultrasound image should be obtained by array.

Output device 130 includes any device capable of displaying or presenting images obtained by a transducer array mounted on or in elongated body 110. For example, output device 130 can include a printer or a CRT monitor.

Navigation system 140 includes any system, apparatus or device capable of tracking or determining a position of a transducer array or distal end 115 of elongated body 110. For example, navigation system 140 can determine a position of an array or distal end 115 using one or more electrical fields and the impedance of the array or catheter, or using magnetism, such as through an Industry Standard Coil Architecture (“ISCA”) system. The position data can include the 3D position (that is, x, y and z) or change in position (that is, Ax, Ay and Az) of the array or distal end 115 of body 110. This position data can be communicated to computing device 120.

Timing system 150 includes any system, apparatus or device capable of measuring a recurring event and reporting the sequence of the recurring event with respect to time to computing device 150. For example, timing system 150 can include a device capable of measuring a rate of repeated cardiac and/or respiratory motion and reporting this rate to computing device 120. In such an example, timing system 150 can include an ECG system.

In another example, timing system 150 can include an imaging device that measures the movement of a patient's diaphragm with respect to time. The imaging device can be, for example, an x-ray imaging device. By measuring diaphragm movement with respect to time, timing system 150 can measure a patient's breathing patterns with respect to time.

In another example, timing system 150 can include expiration sensors. These sensors can measure or calculate the oxygen concentration going in and/or out of a patient's lungs. These measurements or calculations can be used by timing system 150 to determine a patient's breathing patterns with respect to time.

An ultrasound transducer array is mounted proximate distal end 115 of elongated body 110. The array is capable of obtaining 2D ultrasound images in a plurality of imaging planes of a patient anatomy. For example, a linear transducer array capable of obtaining 2D ultrasound images of a patient anatomy can be mounted on or inside distal end 115 of a catheter. Such an array can be manually or mechanically moved or rotated so as to obtain 2D images of a plurality of imaging planes. In another example, a transducer array capable of rotating about the longitudinal axis of elongated body 110 can be mounted on or inside distal end 115 of elongated body 110.

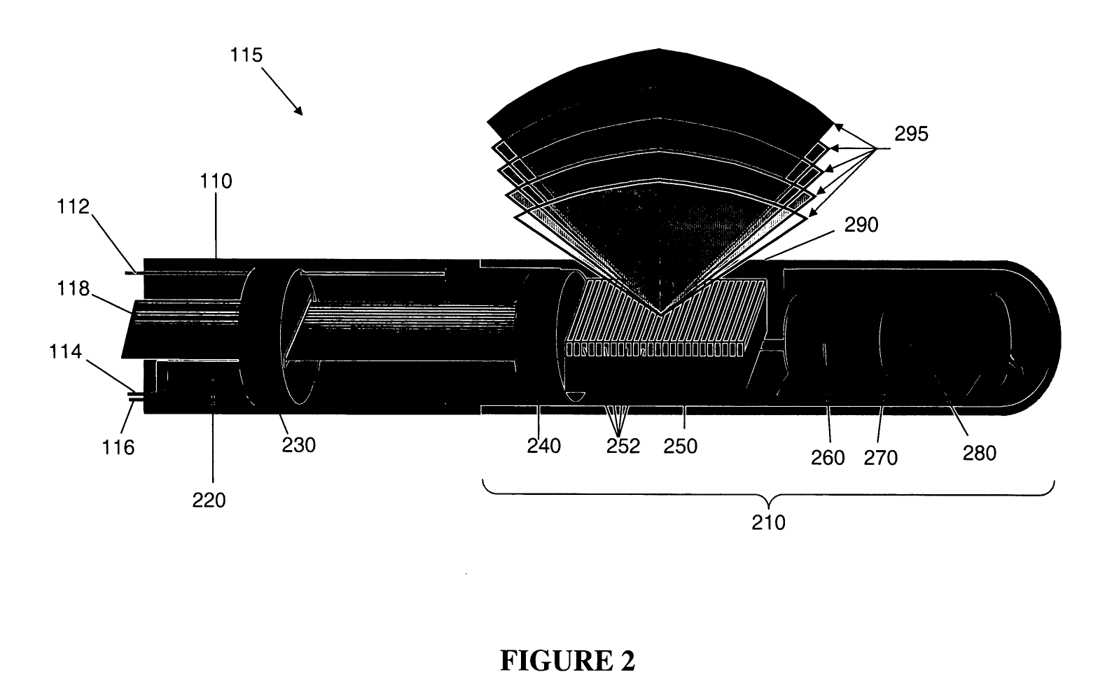

FIG. 2 illustrates distal end 115 of elongated body 110 with a transducer tip 210 in accordance with an embodiment of the presently described technology. FIG. 2 illustrates distal end 115 of elongated body 110 of FIG. 1. Distal end 115 includes a transducer tip 210. Transducer tip 210 can be formed separate from elongated body 110 and subsequently attached or connected to distal end 115 of body 110. In such an embodiment, tip 210 can be fixed to body 110 so that once tip 210 is connected to body 110, tip 210 cannot be removed from body 110. Alternatively, tip 210 can be attached to body 110 in such a way that tip 210 can later be easily removed from body 110.

Alternatively, transducer tip 210 can be an integral part of body 110. That is, tip 210 can be part of body 110 and inseparable from body 110.

In an embodiment, tip 210 can be formed of a combination of materials to ensure that tip 210 is a rigid, non-flexible body. For example, tip 210 can be formed of polyurethane that is surrounded by a polyimide “jacket” that provides the stiffness and rigidity desired for tip 210. Tip 210 can be a rigid, non-flexible body to ensure that tip 210 cannot be bent so as to damage transducer array 250 enclosed therein.

Transducer tip 210 can include a non-rotating seal or bulkhead 230, a cylindrical bearing 240, a transducer array 250 comprising a plurality of ultrasound transducer elements 252, a gearbox 260, a motor 270, a temperature sensor 280 and an ultrasonic output window 290.

In an embodiment of the presently described technology, elongated body 110 includes one or more pull wires/cables 112, a temperature sensor wire/cable 114, a motor wire/cable 116 and one or more transducer communication cables 118. Temperature sensor wire/cable 114 connects temperature sensor 280 to computing device 120 and permits communication of temperature data collected by sensor 280 to device 120. Motor wire/cable 116 connects motor 270 to device 120 and provides a communication path for device 120 to control the rotation of array 250, as described in more detail below. Transducer communication cable(s) 118 connects transducer array 250 to device 120 and provides a communication path for device 120 to cause array 250 to transmit ultrasound beams and for ultrasound echoes received by array 250 to be transmitted to device 120 (as a signal, for example). Computing device 120 can direct array 250 when to obtain an image and/or which 2D plane to image.

In addition, in an embodiment, elongated body 110 and/or tip 210 can include a fluid reservoir 220 to accommodate thermal expansion and/or to compensate for fluid loss during storage of elongated body 110 and/or tip 210.

That is, voids in elongated body 110 and/or tip 210 can include a fluid. Seal 230 can assist in preventing, impeding or stopping fluid from passing from one side of seal 230 to the other.

In an embodiment of the presently described technology, elongated body 110 is a catheter capable of being intravenously steered in a plurality of directions. For example, body 110 can includes a four-way steerable body with a diameter of between 9 and 10 French (−3π mm). Body 110 can be steered using one or more of pull wires/cables 112. In embodiments of the presently described technology, body 110 and tip 210 can be inserted into the cardiac vessels of a patient to obtain ultrasound images.

In an embodiment, transducer array 250 is a one-dimensional (“ID”) array. Array 250 can include several transducer elements 252. For example, array 250 can comprise 64 elements 252 with a pitch of 0.110 nm. That is, array 250 can include a single row of 64 transducer array elements 252. Array elements 252 can be formed of a piezoelectric material. Array 250 can be a linear phased array that operates at a range of frequencies. For example, array 250 can operate at a center frequency of approximately 6.5 MHz, with an operating range of 4-10 MHz.

Window 290 can permit ultrasound beams transmitted by array 250 to pass through tip 210. In an embodiment, window 290 is formed of polyurethane. In another embodiment, window 290 can act as a lens to focus ultrasound beams towards a focal point. That is, window 290 can help to focus ultrasound beams transmitted by array 250. In addition, window 290 can act as a lens to reduce an effect of coupling fluid and the material that tip 210 is formed of on an ultrasound beam transmitted by array 250.

In an embodiment of the presently described technology, array 250 is capable of obtaining a plurality of 2D images 295 in different imaging planes by being rotated about an axis. That is, transducer array 250 can be capable of rotating about the longitudinal axis of tip 210 or elongated body 110 as array 250 transmits and receives ultrasound beams. In an embodiment, array 250 is arranged for oscillatory rotation about the longitudinal axis of tip 210 (that is, back and forth, rather than continuously around). For example, transducer array 250 can obtain 2D image data from a variety of positions as it oscillates about the longitudinal axis of tip 210. One or more hard stops can be placed in tip 210 to limit rotation (that is, prevent 360° rotation about the longitudinal axis of tip 210) and initialize alignment of transducer array 250, window 290, and motor cable/wire 116. In another embodiment, array 250 is arranged for 360° rotation about the longitudinal axis of tip 210. The rotation of array 250 can be limited in radial distance and/or speed. For example, transducer array 250 can be capable of rotating ±30° and obtaining 2D images at 7 vol/second.

Array 250 can be connected to device 120 via cable(s) 118, as described above. Cable(s) 118 can run through all or a portion of tip 210 and/or elongated body 110. In an embodiment, cylindrical bearing 240 can be provided to permit array 250 to rotate without causing communication cables 118 to also be rotated. That is, bearing 240 can permit array 250 to rotate about the longitudinal axis of tip 210 while keeping cables 118 stationary with respect to the longitudinal axis of tip 210.

In an embodiment, motor 270 and gearbox 260 are included in tip 210. For example, motor 270 and gearbox 260 can be located distal to transducer array 250 in tip 250. Control signals sent from device 120 to motor 270 can be used to cause motor 270 to become activated and cause transducer array 250 to rotate, stop array 250 from rotating, or cause array 250 to rotate in the same or different direction. For example, in an embodiment, transducer array 250 and motor 270.

Motion caused by motor 270 can be translated to array 250 via one or more gears in gear box 260. For example, motor 270 can cause one or more gears in gear box 260 to rotate, which in turn cause one or more other gears or the array 250 itself to rotate. In an embodiment, array 250 is connected to a gear in gear box 260 that is rotated to cause rotation of array 250. In another embodiment, a coupling or drive shaft is positioned between motor 270 and gearbox 260 and transducer array 250.

In operation, array 250 obtains a plurality of 2D ultrasound images in a plurality of imaging planes. These images are then combined into a plurality of 3D image segments. The plurality of 3D image segments is then aggregated into a composite 3D image. This composite 3D image can provide more image information than any one of the 3D image segments. For example, the composite 3D image can provide a wider angle of view of a patient anatomy.

Computing device 120 causes transducer array 250 to transmit ultrasound waves to image a plurality of imaging planes. The received ultrasound echoes are communicated from array 250 to device 120 as an electronic signal. Device 120 then forms a 2D image from the received signal. Computing device 120 can cause output device 130 to display or present any one or more of the 2D images. For example, output device 130 can print up a 2D image or display an image on a CRT monitor.

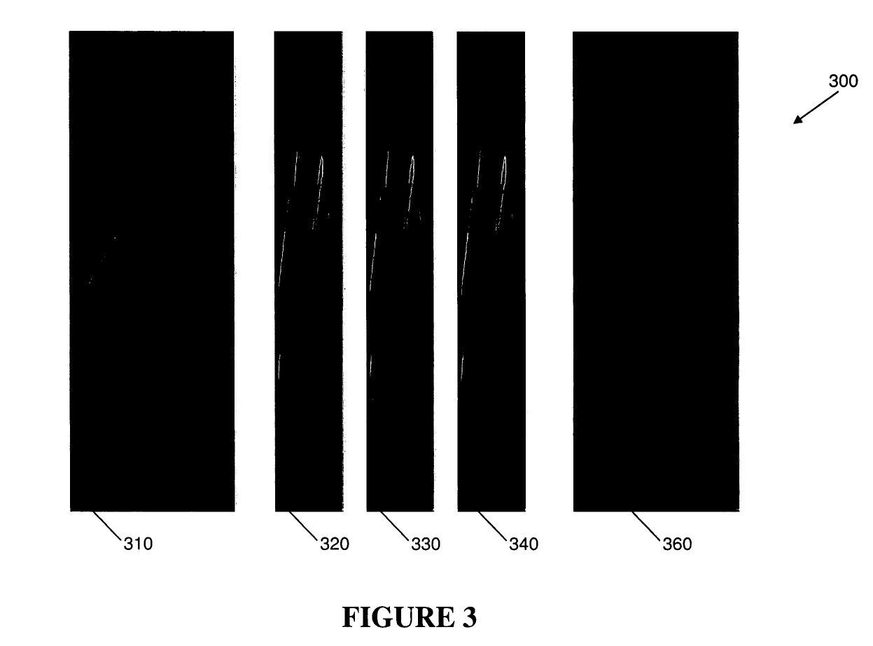

Once at least a plurality of 2D images is obtained from at least a plurality of imaging planes, computing device 120 can combine the 2D images (or image data associated with the 2D images) into at least one 3D image. This 3D image is referred to as a 3D image segment. FIG. 3 illustrates a group 300 of 3D image segments 310, 320, 330, 340, 350, 360 obtained in accordance with an embodiment of the presently described technology. Each of image segments 310-360 is a 3D image segment formed or created by computing device 120 combining a plurality of 2D images.

In an embodiment, computing device 120 combines 2D images into a 3D image segment 310-360 after all 2D images for that 3D image segment 310, 320, 330, 340, 350 or 360 have been obtained. That is, in this embodiment, computing device 120 does not combine the 2D images until all the 2D images are obtained. The 3D image segment 310, 320, 330, 340, 350, 360 is then formed or created in post-image acquisition processing.

In another embodiment, computing device 120 combines or adds 2D images (or image data) to other 2D images or 3D image segments 310, 320, 330, 340, 350, 360 during image acquisition. That is, computing device 120 does not wait for all 2D images to be obtained before combining the 2D images or adding a recently acquired 2D image to a 3D image segment 310, 320, 330, 340, 350, 360. In this way, computing device 120 combines each 2D image with other 2D images or a 3D image segment 310, 320, 330, 340, 350, 360 as each 2D image is obtained.

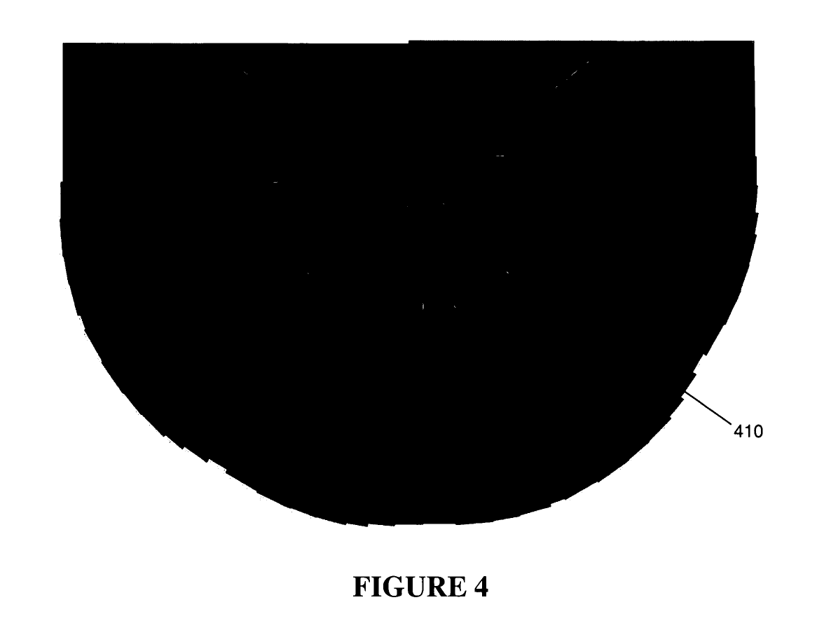

FIG. 4 illustrates a composite 3D image 410 formed or created from a plurality of 3D image segments 310, 320, 330, 340, 350, 360 by computing device 120 in accordance with an embodiment of the presently described technology. Once a plurality of 3D image segments 310, 320, 330, 340, 350, 360 are obtained or formed, computing device 120 aggregates or combines the 3D image segments 310, 320, 330, 340, 350, 360 into one or more composite 3D images 410. While FIG. 3 illustrates five 3D image segments 310, 320, 330, 340, 350, 360, a larger or smaller number of 3D image segments can be combined by computing device 120 to form a composite 3D image 410. For example, as few as two 3D image segments 310, 320, 330, 340, 350, 360, or a number of image segments 310, 320, 330, 340, 350, 360 greater than 5, can be combined by computing device 120 to form a composite 3D image 410.

Computing device 120 can aggregate the 3D image segments 310, 320, 330, 340, 350, 360 into so that a portion of a plurality of the 3D image segments 310, 320, 330, 340, 350, 360 into overlaps one another, for example. In another example, computing device 120 aggregates the 3D image segments 310, 320, 330, 340, 350, 360 into end-to-end. By analogy, this type of aggregation is similar to laying a series of photographs taken of different sections of a horizon next to one another to obtain a full image of the entire horizon. This type of aggregation provides an improvement over existing 3D ultrasound images as the field-of-view of a patient anatomy is considerably greater than 3D images obtained via traditional ultrasound imaging techniques. That is, the anatomical volume represented in composite image 410 is greater than that of any one of image segments 310, 320, 330, 340, 350, 360. For example, as shown in FIG. 4, composite 3D image 410 provides a wider field of view of a patient anatomy than any single one of 3D image segments 310, 320, 330, 340, 350, 360.

Once the 3D composite image 410 is obtained, computing device 120 causes output device 130 to present composite image 410. For example, computing device 120 can cause output device 130 to print out a copy of the composite image 410 or display the composite image 410 on a monitor.

In an embodiment of the presently described technology, computing device 120 combines each of the 3D image segments 310, 320, 330, 340, 350, 360 for a given composite 3D image 410 as each 3D image segment 310, 320, 330, 340, 350, 360 is obtained, or formed by computing device 120. That is, rather than waiting until all 3D image segments 310, 320, 330, 340, 350, 360 for a given composite 3D image 410 are formed before combining them, computing device 120 combines each 3D image segment 310, 320, 330, 340, 350, 360 with other 3D image segments 310, 320, 330, 340, 350, 360 as soon as each 3D image segment is formed.

In an embodiment of the presently described technology, computing device 120 combines the 3D image segments 310, 320, 330, 340, 350, 360 for a given composite 3D image 410 after all 3D image segments 310, 320, 330, 340, 350, 360 are obtained, or formed by computing device 120. That is, rather than combining each 3D image segment 310, 320, 330, 340, 350, 360 with other 3D image segments 310, 320, 330, 340, 350, 360 as soon as each 3D image segment is formed, computing device 120 waits until all 3D image segments 310, 320, 330, 340, 350, 360 for a given composite 3D image 410 are formed before combining them.

In an embodiment of the presently described technology, computing device 120 aligns a plurality of 3D image segments 310, 320, 330, 340, 350, 360 prior to combining the segments into composite 3D image 410. The alignment can include spatial and/or temporal alignment. For spatial alignment, computing device 120 aligns a plurality of 3D image segments 310, 320, 330, 340, 350, 360 to provide the proper spatial layout of segments 310, 320, 330, 340, 350, 360 in composite image 410.

In an embodiment of the presently described technology, spatial alignment can include computing device 120 aligning each of a plurality of 3D image segments 310, 320, 330, 340, 350, 360 with respect to one or more anatomical landmarks imaged in each of the plurality of 3D image segments 310, 320, 330, 340, 350, 360. The anatomical landmarks can be identified by a user of computing device 120. For example, a user can select one or more anatomical landmarks in each 2D image or 3D image segment 310, 320, 330, 340, 350, 360 displayed on output device 130 by computing device 120. Computing device 120 can then align each 3D image segment 310, 320, 330, 340, 350, 360 in composite 3D image 410 by using these user-defined anatomical landmarks. For example, computing device 120 make sure that the same anatomical landmark in adjacent 3D image segments 310, 320, 330, 340, 350, 360 is shown in the same spatial location in composite 3D image 410 by overlapping the adjacent 3D image segments 310, 320, 330, 340, 350, 360.

In another embodiment, spatial alignment can include computing device 120 aligning each of a plurality of 3D image segments 310, 320, 330, 340, 350, 360 with respect to position data of transducer array 250 or tip 210. As described above, position data of transducer array 250 or distal end 115 of elongated body 110 (such as of tip 210, for example) can be obtained by navigation system 140 and communicated to computing device 120. Computing device 120 can associate this position data with 2D images and/or 3D image segments 310, 320, 330, 340, 350, 360. This position data can then be used to provide an accurate spatial layout of each 3D image segment 310, 320, 330, 340, 350, 360 with respect to one another in composite 3D image 410.

For temporal alignment, computing device 120 combines 3D image segments 310, 320, 330, 340, 350, 360 so that image data in each of the combined segments 310, 320, 330, 340, 350, 360 is obtained at an approximately similar time. By approximately similar time, it is meant that the 2D images used to form one or more of image segments 310, 320, 330, 340, 350, 360 are obtained by transducer array 250 within the same time period or within a similar repeated time period. For example, in an embodiment of the presently described technology, timing system 150 can notify computing device 120 of a patient's heart rate and/or breathing patterns with respect to time, as described above. Using this information, computing device 120 can direct array 250 to obtain a 2D ultrasound image at or about the same time. For example, computing device 120 can direct array 250 to obtain a 2D image only when a patient's ECG is at a given peak or valley, or when a patient exhales or inhales (that is, takes a breath). In another embodiment, computing device 120 tracks the time at which each 2D image is obtained by array 250 with respect to a patient's ECG or breathing pattern (provided by timing system 150). Then, in order to temporally align the 3D image segments 310, 320, 330, 340, 350, 360 for a composite 3D image 410, computing device 120 only uses 2D images or 3D image segments 310, 320, 330, 340, 350, 360 to form composite 3D image 410 that were obtained at or at about the same time with respect to a patient's heart beat or breathing pattern.

In an embodiment of the presently described technology, computing device 120 includes a computer-readable storage medium comprising a set of instructions for a computer. The computer-readable storage medium can be embodied in a memory device capable of being read by a computer. The set of instructions can be embodied in one or more sets of computer code and/or software algorithms. The set of instructions includes an aggregation routine. The aggregation routine is configured or written to cause computing device 120 to aggregate a plurality of 3D image segments 310, 320, 330, 340, 350, 360 into a composite 3D image 410 of an anatomy, as described above. The aggregation routine can also be configured to form each of said 3D image segments 310, 320, 330, 340, 350, 360 by combining a plurality of 2D ultrasound images, as described in the various embodiments above.

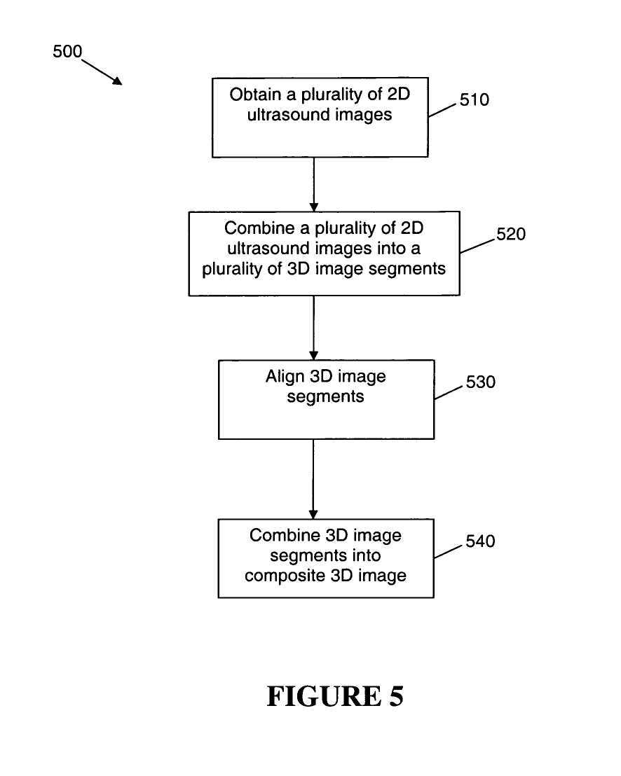

FIG. 5 illustrates a flowchart of a method 500 for aggregating a plurality of 3D image segments into a composite 3D image in accordance with an embodiment of the presently described technology. First, at step 510, a plurality of 2D ultrasound images are obtained, as described above. Next, at step 520, a plurality of the 2D ultrasound images obtained at step 510 are combined to form a plurality of 3D image segments, as described above. Next, at step 530, a plurality of 3D image segments formed or created at step 520 are aligned with respect to time and/or position, as described above. Next at step 540, a plurality of 3D image segments formed at step 520 are combined into a composite 3D image, also as described above.

In an embodiment of the presently described technology, steps 510 and 520 overlap one another. That is, step 510 need not be completed before step 520 is completed. As described above, as each 2D image is obtained, it can be combined to other 2D images or 3D image segments, rather than waiting for all 2D images to be obtained before combining them into a 3D image segment.

In an embodiment of the presently described technology, steps 520 and 530 overlap one another. That is, step 520 need not be completed before step 530 is completed. As described above, as each 3D image segment is formed, it can be aggregated with other 3D image segments, rather than waiting for all 3D image segments to be formed before aggregating them into a 3D composite image. That is, the aggregation of the 3D image segments can be either in real-time (that is, as the image segments are formed) or as part of post processing of retrospective 3D images segments that were acquired previously.

Embodiments of the presently described technology can be used by clinicians in delivering therapy for various procedures and to image cardiac structures. In addition, this technology can also be used in non-medical applications to provide 3D visualizations of internal structures that require an invasive means to reach the structure of interest.

While particular elements, embodiments and applications of the present invention have been shown and described, it is understood that the invention is not limited thereto since modifications may be made by those skilled in the art, particularly in light of the foregoing teaching. It is therefore contemplated by the appended claims to cover such modifications and incorporate those features that come within the spirit and scope of the invention.

What is claimed is:

1. A method for ultrasound imaging, said method including:
obtaining a plurality of ultrasound three-dimensional (“3D”) image segments of an anatomy; and
combining said plurality of 3D image segments into a composite 3D image of said anatomy.

2. The method of claim 1, wherein said obtaining step includes obtaining a plurality of two-dimensional (“2D”) ultrasound images from an ultrasound transducer array mounted proximate a distal end of a catheter and combining said plurality of 2D images into each of said 3D image segments.

3. The method of claim 1, wherein each of said 2D images is combined with other 2D images as each of said 2D images is obtained.

4. The method of claim 1, wherein said plurality of 2D images for a given 3D image segment are combined after all of said plurality of 2D images are obtained.

5. The method of claim 1, wherein said combining step includes combining each of said 3D image segments as each 3D image segment is obtained.

6. The method of claim 1, further including aligning a plurality of said 3D image segments.

7. The method of claim 6, wherein said aligning step includes aligning a plurality of said 3D image segments with respect to time.

8. The method of claim 7, wherein said aligning step includes aligning said plurality of 3D image segments with respect to one or more of cardiac and respiratory motion.

9. The method of claim 1, wherein said composite 3D image represents a greater volume of said anatomy than any one of said 3D image segments.

10. A system for ultrasound imaging, said system including:
a computing device combining a plurality of ultrasound two-dimensional (“2D”) images of an anatomy obtained by a transducer array into one or more three-dimensional (“3D”) image segments and aggregating said 3D image segments into a composite 3D image of said anatomy.

11. The system of claim 10, wherein said transducer array is mounted proximate a distal end of a catheter.

12. The system of claim 10, wherein said computing device combines said 2D images as each of said 2D images is obtained.

13. The system of claim 10, wherein said computing device aggregates said 3D image segments as each 3D image segment is created.

14. The system of claim 10, wherein said computing device aligns a plurality of said 3D image segments.

15. The system of claim 14, wherein said computing device aligns a plurality of said 3D image segments with respect to time.

16. The system of claim 10, wherein said composite 3D image represents a greater volume of said anatomy than any one of said 3D image segments.

17. A computer-readable storage medium comprising a set of instructions for a computing device, said set of instructions including:
an aggregation routine configured to aggregate a plurality of three-dimensional (“3D”) ultrasound image segments into a composite 3D image of an anatomy, said 3D image segments formed by combining a plurality of two-dimensional (“2D”) ultrasound images of an anatomy.

18. The computer-readable storage medium of claim 17, wherein said 2D images are obtained using an ultrasound transducer array mounted proximate a distal end of a catheter and inserted into a heart of a patient.

19. The computer-readable storage medium of claim 17, wherein said aggregation routine combines each of said 2D images with other 2D images as each of said 2D images is obtained.

20. The computer-readable storage medium of claim 17, wherein said aggregation routine aggregates each of said 3D image segments as each 3D image segment is obtained.
